(19) United States
(12) Patent Application Publication
(10) Pub. No.: US 2011/0229408 A1
(43) Pub. Date: Sep. 22, 2011

(54) SODIUM-IODIDE SYMPORTER GENE REPRESSOR BINDING SITE

(76) Inventors: Kenneth AIN, Lexington, KY (US); Wei Li, Lexington, KY (US)

(21) Appl. No.: 12/724,898

(22) Filed: Mar. 16, 2010

(51) Int. Cl. A61K 51/00; C07H 21/00; A61K 31/435; C12Q 1/68; A61P 35/00

(52) U.S. Cl. 424/1.61; 536/22.1; 514/298; 435/6.14

(57) ABSTRACT

The present disclosure relates to a sodium iodide symporter (NIS)-repressor binding sites (NRBS) consensus sequence consisting of a DNA molecule having the sequence 5′-T/C(G/A)GCCT(T/C)A(G/A)TTTCCCCA(T/C)CTGT-3′(the “consensus NRBS”). The disclosure further relates to methods of restoring iodide transport in dedifferentiated thyroid cancer cells by interfering with formation or function of the NIS repressor.

### STATEMENT OF GOVERNMENT SUPPORT

This disclosure was made, in part, with support from the Merit Review award program of the U.S. Department of Veterans Affairs, and the government may have certain rights in this disclosure.

### FIELD OF THE INVENTION

This disclosure relates to nucleotide sequences involved in binding hNIS repressor, kits and methods of restoring iodide transport in cells defective in iodide transport. The present disclosure is further directed to a method of treating tumors by antagonizing the elements that repress the iodide transport in a cancerous cell.

### BACKGROUND OF THE INVENTION

Human sodium-iodide symporter (hNIS) is a trans-membrane protein enabling thyrocytes, both benign and malignant, to concentrate iodide; permitting radioiodine to be a unique systemic cytotoxic therapy for metastatic tumors. Unfortunately, when hNIS expression is lost in dedifferentiated thyroid carcinomas, there are no effective systemic cytotoxic agents (Ain 2000). Restoration of hNIS expression in such tumors could restore effectiveness of radioiodine treatment.

Potential causes for loss of hNIS activity include: hNIS gene mutation (Pohlenz and Refetoff 1999), loss of hNIS gene transcription, defective post-translational processing, and failure to traffic hNIS protein to cell membranes (Pohlenz, et al. 2000). There is no evidence for hNIS gene mutations in thyroid carcinomas. We previously demonstrated that methylation of CpG islands in hNIS promoter can inhibit gene transcription and is reversible with methylation inhibitors (5-azacytidine) and histone deacetylase inhibitors (sodium butyrate) suggesting epigenetic loss of gene expression in thyroid cancer (Venkataraman, et al. 1999). Further investigations revealed evidence for an alternative mechanism for loss of hNIS transcription, suggesting presence of a trans-acting repressor of hNIS transcription, termed NIS-repressor (Li, et al. 2007).

Multiple cellular and nuclear factors are reported to be important for hNIS transcription, including: TSH (thyrotropin)/receptor (TSHr) (Riedel, et al. 2001), TTF-1 (Schmitt, et al. 2001), and Pax-8 (Pasca di Magliano, et al. 2000), but there are no clear examples of repressing transcription factors in thyroid cells or thyroid carcinomas. In U.S. application 60/907,881, we showed NIS-repressor as a trans-acting protein binding to a specific region of the proximal hNIS promoter, NIS-repressor binding site (NRBS-P); however its composition was not yet known. We also characterized NIS-repressor and investigated the identities of its components and mechanisms of its activity. This involved defining NRBS-P to a narrower region of hNIS promoter and utilizing it to probe nuclear extract, analyzing the probe-bound proteins with liquid chromatography coupled with tandem mass spectrometry (LC/MS/MS), to characterize NIS-repressor components. The mass spectrometry analysis data demonstrated human Poly (ADP-ribose) polymerase 1 (PARP-1) to be a likely component of the NIS-repressor protein complex. Pharmacological inhibition of PARP-1 activity with PJ34, a Parp inhibitor, stimulated endogenous hNIS mRNA levels, providing evidence that PARP-1 acts as a negative regulatory factor for hNIS transcription and is a likely component of the NIS-repressor complex.

Because of its role in inhibiting the transport of iodide into cells, and in particular, into thyroid cancer cells, there is a need to determine the hNIS repressor binding sites, structure and activities so that anti-thyroid cancer therapies can be maximized.

### SUMMARY OF THE INVENTION

One aspect of the invention relates to a sodium iodide symporter (NIS)-repressor binding site (NRBS) consisting of a DNA molecule spanning from −1067 to −868 (SEQ ID NO.: 2). Another aspect of the invention relates to a sodium iodide symporter (NIS)-repressor binding site (NRBS) consisting of a DNA molecule having the sequence 5′-TG(G/A)GCCT(T/C)A(G/A)TTTCCCCA(T/C)CTGT-3′ (SEQ ID NO.: 1) or a nucleotide sequence that hybridizes to the complement thereof under high stringency conditions.

Yet another aspect of the invention relates to a method of treating thyroid cancer comprising administering to a patient in need thereof a therapeutically effective amount of a PARP-1 inhibitor and a therapeutically effective amount of radiolabeled iodide. In an other aspect of the invention there is provided a method of treating thyroid cancer in a patient comprising contacting thyroid cancer cells in the patient that express and form a NIS repressor protein complex capable of binding to SEQ ID NO.: 1 or SEQ ID NO.: 2 with a PARP-1 inhibitor, and administering to the cells radiolabeled iodide.

### BRIEF DESCRIPTION OF THE DRAWINGS

FIGS. 1A and B show the results of EMSA analysis followed by SDS electrophoresis to find additional binding sites for NIS-repressor. In FIG. 1A radiolabeled Probe-A, radiolabeled SHIFT-1, radiolabeled SHIFT-2, radiolabeled SHIFT-3 were used in lanes 1 to 3, 4 to 6, 7 to 9, and 10 to 12, respectively. Lanes 1, 4, 7, and 10 contain the respective labeled probes only. KAK1 nuclear extract is included in all other lanes, with lanes 3, 6, 9, and 12 containing 30× unlabeled respective probe. In FIG. 1B, radiolabeled Probe-A, radiolabeled SHIFT-4, and radiolabeled SHIFT-5, are used in lanes 1 to 3, 4 to 6, and 7 to 9, respectively. Lanes 1, 4, and 7 contain the respective hot probes only. KAK1 nuclear extract is included in all other lanes, with lanes 3, 6, and 9 containing 30× unlabeled respective probe. The arrows point to probe-specific bands.

FIGS. 2A and 2B show the results of EMSA analysis followed by SDS gel electrophoresis to define the core sequence for NRBS-D and cross competition of NRBS-D with NRBS-P. FIG. 2A depicts EMSA using KAK1 nuclear extract probed with radiolabeled SHIFT-4 containing NRBS-D in lanes 2 to 15. Unlabeled (30×) SHIFT-4, 4.1, 4.4, 4.2, 4.3, 4.5, 4.6, 4.7, and Probe-A were included in EMSA reactions in lanes 3, 4, 5, 6, 7, 11, 12, 13, and 15, respectively. The unlabeled (60×) annealed double-stranded oligonucleotides ds-411, ds-412, ds-413, and Comp-1 were added to the EMSA reactions in lanes 8, 9, 10, and 14, respectively. Lane 2 had no additional unlabeled competitor, and lane 1 contained radiolabeled SHIFT-4 probe only. In FIG. 2B, KAK1 nuclear extract was probed with radiolabeled Probe-A. The unlabeled 30× Probe-A, 60× annealed double-stranded Comp-1, and 60× annealed double-stranded ds-414 were added in EMSA reactions in lane 3, 4, and 5, respectively. The arrows point to the probe-specific bands.

FIGS. 3A, B and C show the results of a supershift experiment in which antibodies against thyroid-related transcription factors using Cal-62 nuclear extract with a probe that contains NRBS-P (bp −653 to −615) or NRBS-D. Experiments depicted in 3A and 3B were performed using Comp-1 probe while experiments in 3C used SHIFT-414 probe. In all three sections Lane 1 contains probe only with all other lanes containing basal Cal-62 nuclear extract and Lane 3 contains 50× cold respective probes. In 3A, specific antibodies were added to respective lanes as follows: Lane 4, anti-TTF-1; Lane 5, anti-TTF-2 (S-18); Lane 6, anti-Pax8; Lane 7, anti-Sp1; Lane 8, anti-c-Jun; Lane 9, anti-c-Fos; Lane 10, anti-AP2α; and Lane 11, anti-PARP-1. In both 3B and 3C, specific antibodies were added as follows: Lane 4, anti-TTF-2 (S-18); Lane 5, anti-TTF-2 (F-17); and Lane 6, anti-TTF-2 (V-20).

### DETAILED DESCRIPTION

Radioiodine therapy remains the only known effective systemic tumoricidal treatment for thyroid carcinoma. Unfortunately, around 10% of such cancers and most dedifferentiated thyroid cancers fail to concentrate radioiodine consequent to loss of sodium-iodide symporter gene (NIS) expression (Ain 2000; Robbins, et al. 1991). For that reason, efforts to understand the mechanisms of this loss may lead to new treatments to restore NIS expression, permitting effective therapy with radioiodine. Our previous study provided evidence of a trans-active protein factor (complex) suppressing NIS transcription under basal conditions, possibly accounting for loss of human NIS expression in some thyroid cancers. This suggested a new target, which we named NIS-repressor, for designing therapies to restore radioiodine uptake in disseminated tumors. We mapped its binding-site in the proximal NIS promoter (NIS-repressor binding site; NRBS-P) (Li et al. 2007). This repressor may function in concert with or independent of epigenetic effects on NIS expression via NIS promoter methylation and histone deacetylation (Venkataraman et al. 1999).

The present invention is based, in part, on the identification of a second site in the human sodium-iodide symporter (NIS) promoter region, herein, referred to as NIS-repressor binding site (NRBS-D). We further investigated NIS-repressor by refining NRBS-P, demonstrating sequences at −648 to −620 bp, and an additional NRBS at −987 to −958 by (NRBS-D; relative to the NIS translation start site) as two core binding sites for NIS-repressor. The homology between NRBS-D and NRBS-P core sequences is 83% in a 23 by region, with two A/G and two T/C transitions. This 23 by consensus sequence (5′-TG(G/A)GCCT(T/C)A(G/A)TTTCCCCA(T/C)CTGT-3′) (SEQ ID NO.: 1)(“consensus NRBS”) is in opposite orientation between NRBS-P and NRBS-D in the hNIS promoter and 310 by apart from each other. A human genome homology search (NCBI/BLAST/blastn suite) shows this consensus sequence to occur (at >90% homology) within two kilobases of the 5′-end of more than 20 different genes in the human genome. Among these genes, there are some coding for kinases, receptors, and transporters.

EMSA analysis showed proteins in KAK1 nuclear extract that bound to NRBS-P and constitute the NIS-repressor. Electrophoretic analysis of these nuclear extract proteins, UV-crosslinked to the radiolabeled NRBS-P probe, revealed multiple bands, suggesting that NIS-repressor is a protein complex. Several thyroidal transcription factors (Sp1, Ap1, AP2, TTF-1 and Pax8), previously characterized as affecting NIS transcription, were excluded as candidates for NIS-repressor components because double-stranded oligonucleotides containing their respective consensus DNA-binding sites failed to compete against a radiolabeled NRBS-P probe in EMSA analysis.

Unexpectedly, an antibody against human thyroid transcription factor 2 (hTTF-2) (antibody S-18), but not two other anti-TTF-2 antibodies (F-17 or V-20), which recognize different epitopes on TTF-2, altered the migration of the probe-protein complex in supershift assays, demonstrating that human TTF-2 is associated with, or is a part of, the NIS-repressor complex. The three tested antibodies are available from Santa Cruz Biotechnology, Inc. S-18 is an affinity purified goat polyclonal antibody raised against a peptide mapping within an internal region of the human TTF2 polypeptide. The epitope for this antibody is the region from amino acid 100-150 in human TTF2. F-17 is an affinity purified goat polyclonal antibody raised against a peptide mapping within an internal region of human TTF2. The epitope for this antibody is the region from amino acid 140-190 in human TTF2, and S-18 and F-17 do not have competing binding sites. V-20 is an affinity purified goat polyclonal antibody raised against a peptide mapping near the C-terminus of human TTF2.

In one aspect of the invention, an inhibitor of TTF-2 is administered to a patient suffering from thyroid cancer to inhibit the formation of the NIS-repressor complex and/or binding of the NIS repressor to either or both of NRBS-P and NRBS-D and restore iodide uptake in dedifferentiated thyroid carcinoma cells.

Although 5-azacytidine and sodium butyrate have been shown to restore NIS transcription (Venkataraman et al. 1999), these agents did not alter the EMSA pattern using KAK1 nuclear extract, suggesting that NIS-repressor represents a different mechanism of NIS gene regulation. This is consistent with our previous genomic DNase I digestion studies (Li et al. 2007) that failed to demonstrate any effect of these agents on chromatin compaction, suggesting the possibility of non-epigenetic regulatory processes.

The human poly(ADP-ribose) polymerase-1 (PARP-1; EC 2.4.2.30) was identified by proteomic analysis of the nuclear extract from KAK1 cells, as a top candidate for a component of the NIS-repressor complex. PARP-1 was initially known for its role as a DNA-damage sensor, repair and signaling protein. Later studies have shown that PARP-1 also participates in additional critical cellular activities, such as: apoptosis, genetic stability, and gene transcription (Schreiber, et al. 2006). PARP-1 was reported to be able to bind to regulatory sequences by itself (Chiba-Falek, et al. 2005; Zhang, et al. 2002), modify some transcription factors or signal proteins by poly(ADP-ribosyl)ation (Miyamoto, et al. 1999), and influence other protein factors by hetero-complex formation (Simbulan-Rosenthal, et al. 2003). A recent study reveals that PARP-1 has widespread effects upon transcription of diverse genes, either as a positive or negative transcription factor (Krishnakumar, et al. 2008).

ChIP analysis of KAK1 cells with two commercial anti-PARP-1 antibodies shows that PARP-1 is associated with the NRBS-P region in KAK1 cells under basal culture conditions without NIS transcription. Furthermore, PJ34, an inhibitor of PARP-1 enzymatic activity (Abdelkarim et al. 2001), effectively stimulated luciferase activity from NIS promoter constructs and also stimulated endogenous hNIS transcription in both KAK1 and Cal-62 cells, confirming that PARP-1 is part of a negative regulatory factor for hNIS gene transcription. Despite the ChIP data indicating that PARP-1 was associated with the hNIS promoter region containing NRBS-P, two different commercial anti-hPARP-1 polyclonal antibodies (that had been effective in the ChIP assay) failed to alter the EMSA pattern on supershift analysis. In addition, two commercial preparations of human PARP-1 failed to produce the same EMSA signals as the nuclear extract from KAK1 cells. It is likely that PARP1 does not directly bind to the NRBS sequence; rather, it is associated with other proteins that contain the critical DNA-binding domain. PJ34 inhibition of PARP1 enzymatic activity may compromise the assembly, stability, or activity of the NIS-repressor protein complex.

In summary, a second core sequence in the human sodium-iodide symporter (hNIS) promoter, NRBS-D, which is a binding site for a trans-active transcriptional repressor, NIS-repressor has been defined. Proteomic analysis revealed PARP-1 as an important constituent of the NIS-repressor protein complex. A known inhibitor of PARP-1 enzymatic activity, PJ34, causes increased endogenous transcription of hNIS in genotypically verified thyroid cancer cells.

In one aspect of the invention there is provided a method of screening for therapeutic agents capable of restoring NIS gene expression and radioiodine uptake in thyroid cancer cells. The method comprises the steps of: i) contacting thyroid cancer cells with a pharmacologic antagonist against one or more components of the NIS repressor protein complex capable of binding to SEQ ID NO. 1, ii) detecting NIS expression or radioiodine uptake by the cell; and iii) selecting the pharmacologic antagonist that results in an increase in NIS expression or radioiodine uptake by the thyroid cancer cells. In certain embodiments the pharmacologic antagonist is an inhibitor of PARP-1 or TTF-2, wherein inhibition thereof comprises inhibition of NIS complex binding to SEQ ID NO. 1 or inhibition of NIS complex formation or function.

The 23 base pair NRBS consensus sequence (SEQ ID NO.: 1) may have regulatory importance for multiple diverse human genes. In thyroid oncology, NIS-repressor is a useful target in restoring the effectiveness of radioiodine therapy to dedifferentiated thyroid cancers.

Additional objects and advantages of the disclosure will be set forth in part in the description which follows, and in part will be obvious from the description, or may be learned by practice of the disclosure.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and H (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.).

As used herein “stringent hybridization conditions” are generally selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. High stringency conditions are selected to be equal to the Tₘ point for a particular probe. An extensive guide to the hybridization of nucleic acids is found in Tijssen (1993) Laboratory Techniques in Biochemistry and Molecular Biology, incorporated herein in its entirety.

The following describes materials and methods used in the procedures described in the subsequent Examples.

### EXAMPLES

### Example 1

### A Second NRBS (NRBS-D) in the hNIS Promoter

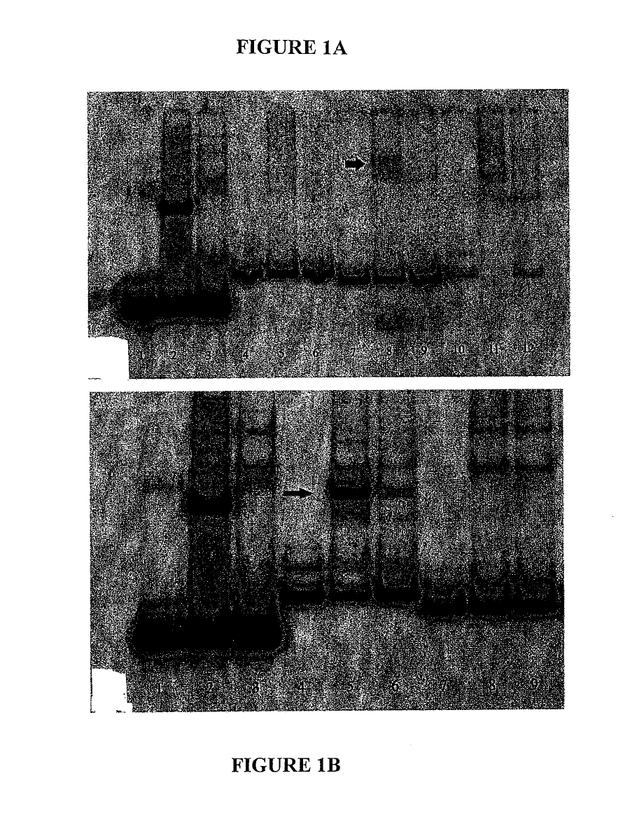

Five EMSA probes, SHIFT-1, -2, -3, -4, and -5, were prepared with PCR, radiolabeled, and used to probe KAK1 nuclear extract in EMSA. The EMSA results shown in FIG. 1 indicate that: 1) no specific signal for SHIFT-1 probe (FIG. 1A, lane 5), covering −1667 to −1468 bp; 2) multiple faint specific signals for SHIFT-2 probe (FIG. 1A, lane 8), covering −1467 to −1268 bp; 3) no specific signal for SHIFT-3 probe (FIG. 1A, lane 11), covering −1267 to −1068 bp; 4) one strong specific signal for SHIFT-4 probe (FIG. 1B, lane 5), covering −1067 to −868 bp; and 5) no specific signal for SHIFT-5 probe (FIG. 1B, lane 8), covering −873 to −708 bp. This shows that KAK1 nuclear extract contains one or more factors that can bind to the sequence from −1067 to −868 by in the hNIS promoter, further upstream from NRBS-P. We designate this region as a distal NRBS (NRBS-D).

### Example 2

### The Core Sequence of NRBS-D is Homologous to NRBS-P and demonstrates Cross-Competition Between Both Sites

Seven PCR fragments and three annealed double-strand oligonucleotides were used as unlabeled competitors against the radiolabeled SHIFT-4 probe in EMSA to determine the core sequence for NRBS-D. The seven PCR fragments are: SHIFT-4.1 (150 bp; −1017 to −868), SHIFT-4.2 (100 bp; −967 to −868), SHIFT-4.3 (150 bp; −1067 to −918), SHIFT-4.4 (100 bp; −1017 to −918), SHIFT-4.5 (150 bp; −1017 to −868), SHIFT-4.6 (140 bp; −1007 to −868), and SHIFT-4.7 (130 bp; −997 to −868). The three annealed double-stranded oligonucleotides are: ds-411 (5′-tttattcctctgaggcagggtctattttat-3′, 30 bp; −1017 to −988) (SEQ ID NO.: 3), ds-412 (5′-tgaggcagggtctattttatccttgttaca-3′, 30 bp; −1007 to −978) (SEQ ID NO.: 4), and ds-413 (5′-tctattttatccttgttacagatggggaaa-3′, 30 bp; −997 to −968) (SEQ ID NO.: 5). Only the sequences of the sense strands are listed. Probe-A and annealed double-stranded Comp-1 were also included as cold competitors, as we considered NRBS-D to be an additional binding site for NIS-repressor, which had already been demonstrated to bind to NRBS-P.

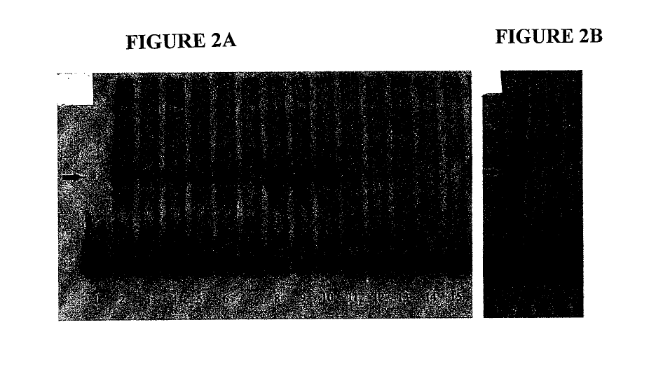

These EMSA results are shown in FIG. 2, revealing that all three annealed double-stranded oligonucleotides (ds-411, ds-412, and ds-413) do not compete against the radiolabeled SHIFT-4 probe (FIG. 2A, lanes 8-10) and that the SHIFT-4.2 fragment does not compete against this probe either (FIG. 2A, lane 6). All of the other PCR fragments (SHIFT-4.1, SHIFT-4.4, SHIFT-4.3, SHIFT-4.5, SHIFT-4.6, and SHIFT-4.7) compete effectively against the radiolabeled SHIFT-4 probe (FIG. 2A, lanes 4, 5, 7, 11-13). The unlabeled Probe-A (FIG. 2A, lane 15) and the unlabeled double-stranded oligonucleotide, Comp-1 (FIG. 2A, lane 14), strongly compete against the same probe. These data suggest that the sequence around −1017 to −968 by is critical for the effects of NRBS-D and the NIS-repressor binding to NRBS-P can also bind to NRBS-D.

Further analysis, using an unlabeled annealed double-stranded oligonucleotide (ds-414; 5′-ccttgttacagatggggaaactaaggccca-3′, 30 bp; −987 to −958) (SEQ ID NO.: 6), sharing a 20 by sequence with NRBS-D and having an additional unshared 10 by sequence downstream, revealed strong competition against the radiolabeled Probe-A in EMSA (FIG. 2B, lane 5). This suggests that the NIS-repressor, binding to NRBS-D, can also bind to the NRBS-P. Thus, NRBS-D and NRBS-P can cross-compete efficiently against each other in EMSA, indicating that NIS-repressor, in KAK1 nuclear extract, can bind to either NRBS-P or NRBS-D in the hNIS promoter region.

### Example 3

### Association of TTF-2 with NRBS-P and NRBS-D

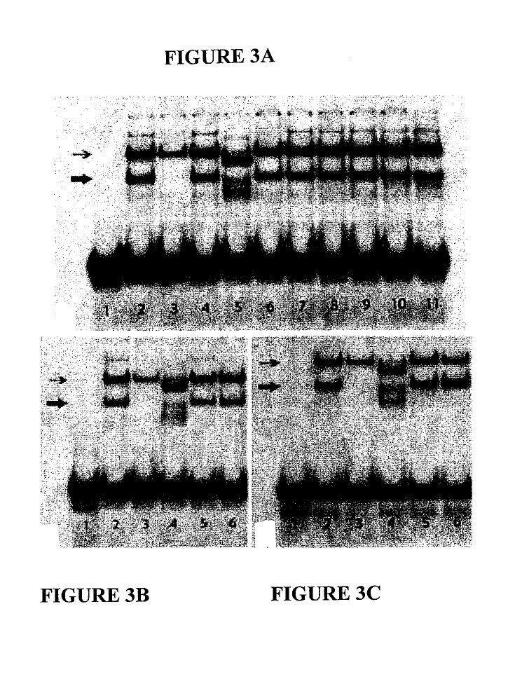

In supershift assays, antibodies against human Sp1 (E-3), c-Jun (H-79), c-Fos (H-125), AP-2a (C-18), TTF-1 (F-12), Pax8 (A-15), and PARP-1 failed to alter the EMSA signal mobilities, suggesting that their respective antigens are not associated with the NRBS-P site. This is consistent with other results showing that their respective consensus DNA target sequences are unable to compete against NRBS-P. The anti-TTF-2 antibody (S-18) shifted the EMSA signals, changing the mobility of one of the bands, showing faster migration, and simultaneously changing the single Comp-1 specific signal into multiple constituent bands with faster migration on the gel, as shown in FIG. 3A, lane 5. We attempted to further verify this phenomenon with two additional anti-TTF-2 commercial antibodies, recognizing different TTF-2 epitopes. Both of these antibodies (F-17, V-20) failed to alter the EMSA signals as achieved with the S-18 antibody. This indicates that TTF-2 is a constituent of the protein factors responsible for the EMSA signals with NRBS-P (FIG. 3B) and NRBS-D probes (FIG. 3C), demonstrating that human TTF-2 is likely to be part of the NIS-repressor complex.

1. An isolated nucleic acid sequence having the sequence 5″-TG (G/A)GCCT(T/C)A(G/A)TTTCCCCA(T/C)CTGT-3′ (SEQ ID NO.: 1) or a nucleotide sequence that hybridizes to the complement thereof under high stringency conditions.

2. A method of treating thyroid cancer comprising administering to a patient in need thereof a therapeutically effective amount of a Poly (ADP-ribose) polymerase 1 (PARP-1) inhibitor and a therapeutically effective amount of radiolabeled iodine.

3. The method of claim 2 wherein the PARP-1 inhibitor is PJ34.

4. The method of claim 2 wherein the thyroid cancer is resistant to treatment with radiolabeled iodine alone.

5. An isolated nucleotide sequence consisting of the sequence from −1067 to −868 base pairs upstream of the translation start site of human sodium iodide symporter gene (SEQ ID NO.: 2).

6. A method of treating thyroid cancer in a patient comprising contacting thyroid cancer cells in the patient that express and form a NIS repressor protein complex capable of binding to SEQ ID NO. 1 or SEQ ID NO. 2 with a PARP-1 inhibitor, and administering to the cells radiolabeled iodine.

7. A method of treating thyroid cancer in a patient comprising administering to the patient an inhibitor of human sodium-iodide symporter (hNIS) repressor complex formation or an inhibitor of hNIS repressor function.

8. The method of claim 7 wherein the inhibitor is a PARP-1 inhibitor.

9. A method of screening for a therapeutic agent capable of restoring NIS gene expression and radioiodine uptake in a thyroid cancer cell, comprising: the steps of: i) contacting the cell with a pharmacologic antagonist against one or more components of an NIS repressor protein complex capable of binding to SEQ ID NO. 1, ii) detecting NIS expression or radioiodine uptake by the cell; wherein an increase in the NIS expression or radioiodine uptake by the cell indicates that said agent is capable of restoring radioiodine uptake in thyroid cancer cells.


SEQUENCE LISTING (6 sequences)

SEQ ID NO: 1 - DNA, 23 bp, Homo sapiens
  misc_feature (3)..(3): transition from g to a.
tgggccttagtttccccatctgt

SEQ ID NO: 2 - DNA, 200 bp, Homo sapiens
  misc_feature (1)..(200): The sequence  from-1067 to -868 of human NIS promoter with A in ATG of hNIS is designated as +1
taaatgaatgaggcacttatcatgtaccaggctttcgttaccacgtcccatttattcctctgaggcagggtctattttatccttgttacagatggggaaactaaggcccagggaggagcaaagtcttccccaagtatgtacccactcagaacttgagctctgaatgtctcccacccagcttagcccaagagcggggttca

SEQ ID NO: 3 - DNA, 30 bp, Homo sapiens
tttattcctctgaggcagggtctattttat

SEQ ID NO: 4 - DNA, 30 bp, Homo sapiens
tgaggcagggtctattttatccttgttaca

SEQ ID NO: 5 - DNA, 30 bp, Homo sapiens
tctattttatccttgttacagatggggaaa

SEQ ID NO: 6 - DNA, 30 bp, Homo sapiens
ccttgttacagatggggaaactaaggccca